(19) United States
(12) Patent Application Publication
(10) Pub. No.: US 2002/0178489 A1
(43) Pub. Date: Dec. 5, 2002

(54) PAD STRUCTURE OF SWIMMING GLASSES

(76) Inventor: Hsiu-Ying Sung, Taipei (TW)

Correspondence Address: ROSENBERG, KLEIN & LEE, 3458 ELLICOTT CENTER DRIVE - SUITE 101, ELLICOTT CITY, MD, 21043, US

(21) Appl. No.: 09/872,432

(22) Filed: May 30, 2001

(51) Int. Cl. A61F 9/02

(52) U.S. Cl. 2/428

(57) ABSTRACT

The present invention proposes an improved pad structure of swimming glasses, wherein the pad thereof is integrally formed of soft material. The outer side of the pad forms a ring face, which can contact tightly with the skin of eye socket to achieve airtight effect. A plurality of enhancement structures for increasing the resiliency of the pad are alternately disposed between the inner and outer side faces of the pad, thereby effectively resisting the pressure of water and increasing the resiliency of the swimming glasses. Moreover, the position of the swimming glasses can be elevated to prevent the nose bridge from contacting with a hard joining ring and to enhance the comfort of wearing.

### FIELD OF THE INVENTION

The present invention relates to an improved pad structure of swimming glasses and, more particularly, to a waterproof device worn when swimming to avoid discomfort of eyes.

### BACKGROUND OF THE INVENTION

As shown in FIGS. 1 to 3, a pair of swimming glasses in the prior art comprise two lenses 50 made by extrusion of hard and transparent material. The adjacent sides of the two lenses 50 extend to form two lock rings 51, and the left and right sides thereof also extend to form two lock rings 51a, respectively. A joining ring 52 is disposed between the two lenses 50. The joining ring 52 can connect the two lock rings 51 so that the two lenses 50 can be joined together. A plurality of flanges 52a are disposed at inner sides of two ends of the joining ring 52. The flanges 52a can be locked with the lock rings 51 to adjust the distance between the two lenses 50. The two lock rings 51a at the left and right sides of the two lenses 50 are connected by a head band 53. The inner side of the lens 50 forms a ring face 50a. The ring face 50a at the inner side of the lens 50 joins a pad 54. A groove 54a is formed in the midst of the pad 54. The outer edge of the groove 54a also has a ring face 55.

Please also refer to FIGS. 1 to 3, when the swimming glasses are worn, the head band 53 of the swimming glasses is sleeved on the head and fixed, the two lenses 50 made by extrusion of hard and transparent material are then adjusted and fixed on the skins of eye sockets, and the ring faces 55 of the pads 54 contact tightly with the skins of eye sockets, thereby letting the swimming glasses achieve airtight effect.

However, when the swimming glasses are worn, it is necessary to pull the head band 53 to the head and fix it and let the ring faces 55 of the pads 54 contact tightly with the skins of eye sockets. For comfort of wearing, the pad 54 is made of soft material. Because the material of the groove 54a is also soft material, and the soft material near the eye socket is very thin, the groove 54a easily shrinks and the material easily deforms when overly used, hence losing the airtight effect.

Moreover, because of shrinkage and deformation of the groove 54a, the position of the swimming glasses will be too near the face so that the nose bridge of the user will contact with the hard joining ring 52 of the swimming glasses, resulting in discomfort or even abrasion of the skin of the nose bridge.

When the user swims, the water pressure will press the swimming glasses to shrink the grooves 54a, resulting in bad circulation of blood. Therefore, the swimming glasses cannot be worn for a long time. Moreover, when the swimming glasses are taken off, pressed marks will be generated on the face because of bad circulation of blood.

Therefore, the above pad structure of swimming glasses has inconvenience and drawbacks in practical wearing or use. The present invention aims to resolve the above problems in the prior art.

### SUMMARY OF THE INVENTION

The primary object of the present invention is to provide an improved pad structure of swimming glasses capable of effectively increasing the resiliency of swimming glasses and enhancing the comfort of wearing.

The secondary object of the present invention is to provide an improved pad structure of swimming glasses so that pressed marks generated on the skins of eye sockets because of bad circulation of blood can be avoided.

Another object of the present invention is to provide an improved pad structure of swimming glasses, wherein a plurality of enhancement structures are added and alternately disposed between the inner and outer side faces of the pad to elevate the position of the swimming glasses and to prevent the abrasion of the skin of the nose bridge because of the contact of the nose bridge with the hard joining ring of the swimming glasses.

The various objects and advantages of the present invention will be more readily understood from the following detailed description when read in conjunction with the appended drawing, in which:

### BRIEF DESCRIPTION OF THE DRAWINGS

FIG. 1 is a perspective view of a pair of swimming glasses in the prior art;

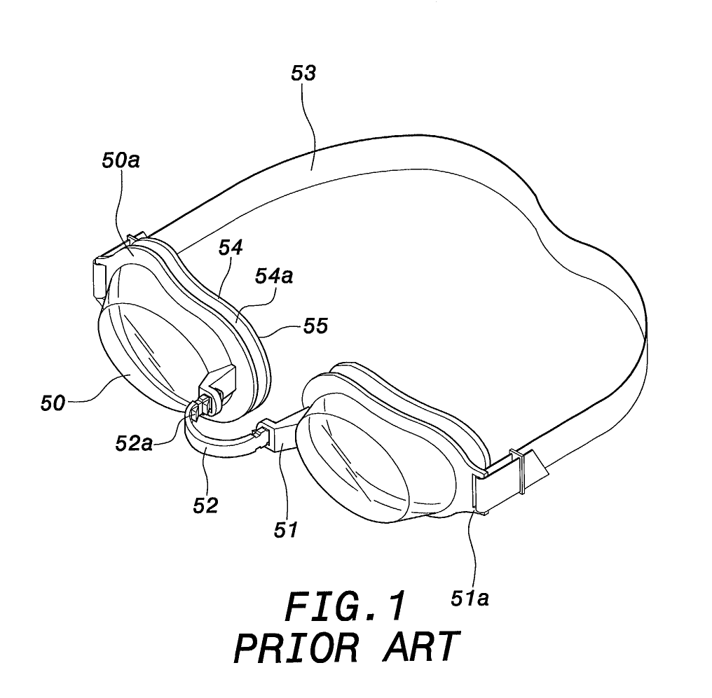

FIG. 2 is a top view of a pair of swimming glasses in the prior art;

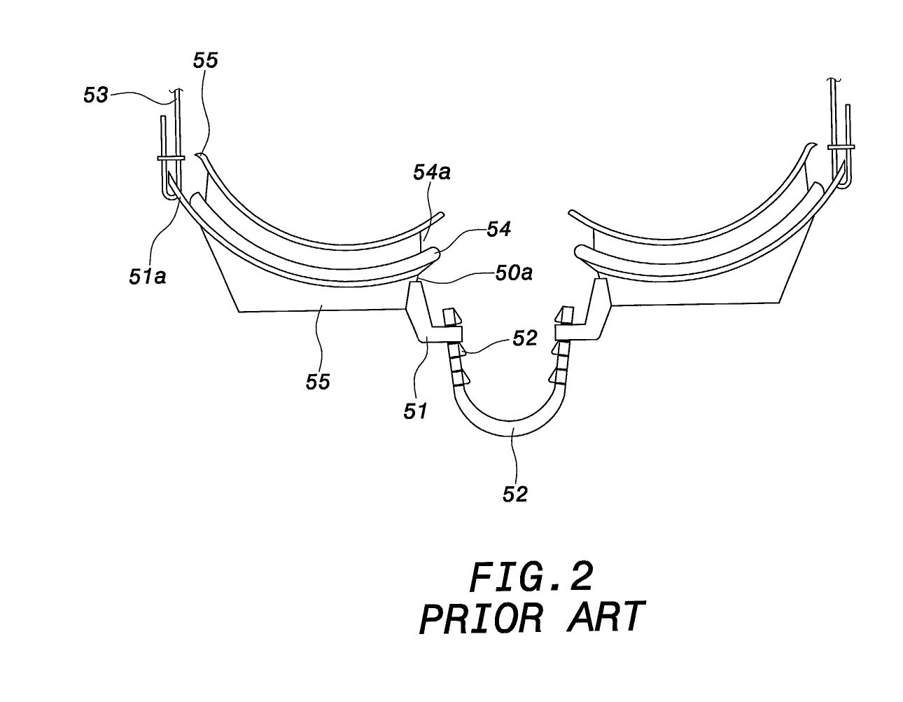

FIG. 3 is a cross-sectional view showing the use state of a pair of swimming glasses in the prior art;

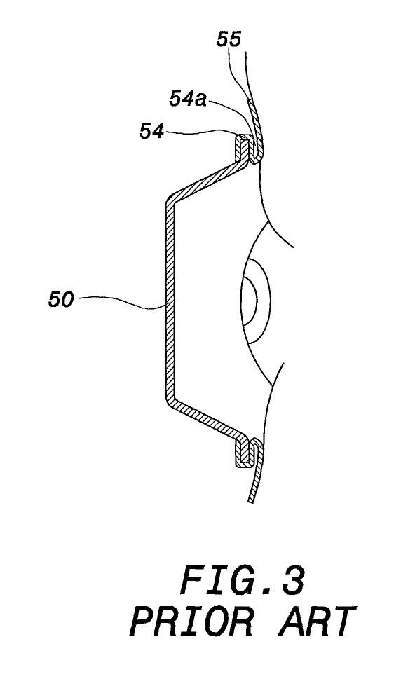

FIG. 4 is a perspective view of an improved pad structure of swimming glasses of the present invention;

FIG. 5 is a top view of an improved pad structure of swimming glasses of the present invention;

FIG. 6 is a cross-sectional view of an improved pad structure of swimming glasses of the present invention; and

FIG. 7 is a cross-sectional view showing the use state of an improved pad structure of swimming glasses of the present invention.

### DETAILED DESCRIPTION OF THE PREFERRED EMBODIMENTS

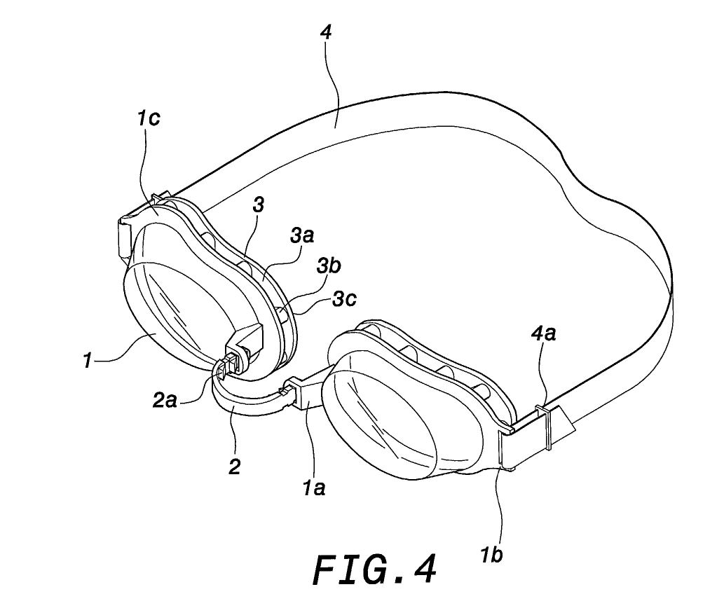

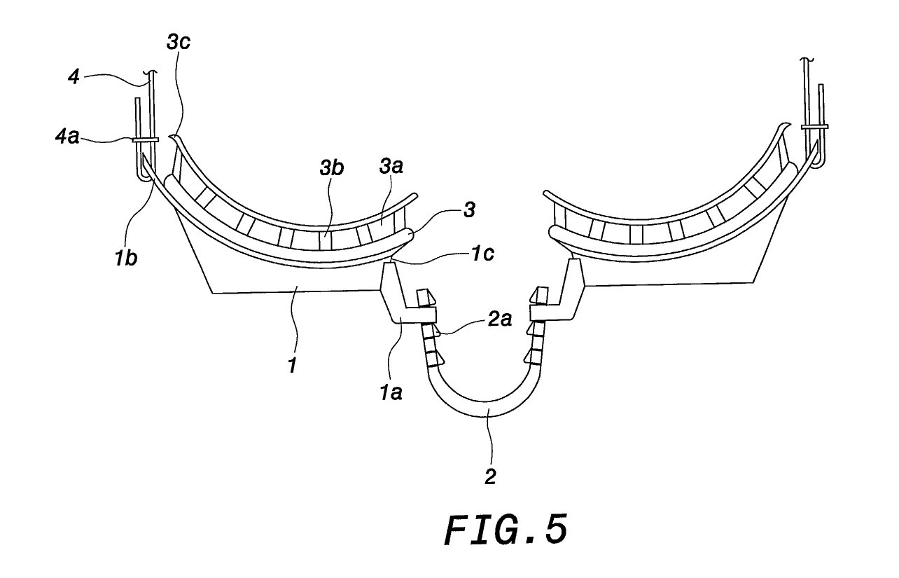

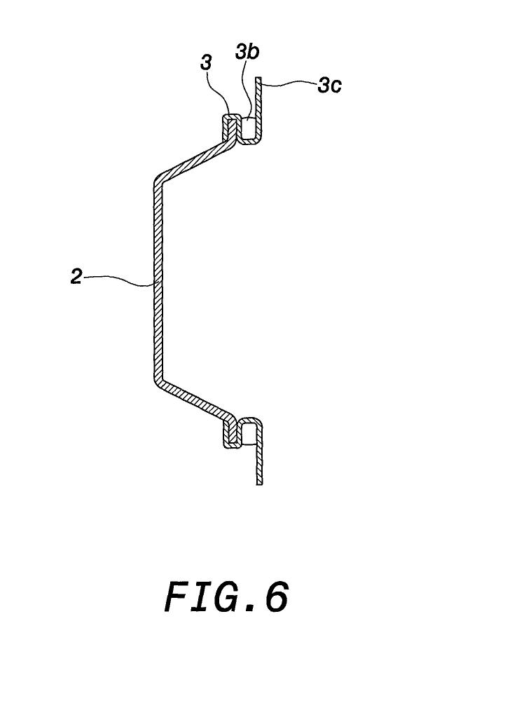

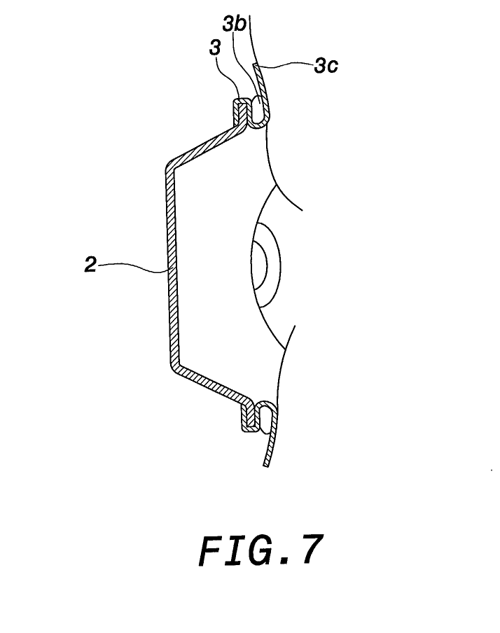

As shown in FIGS. 4 to 7, an improved pad structure of swimming glasses of the present invention comprises two lenses 1 made by extrusion of hard and transparent material. The adjacent sides of the two lenses 1 extend to form two lock rings 1a, and the left and right sides thereof also extend to form two lock rings 1b, respectively. A joining ring 2 is disposed between the two lenses 1. The joining ring 2 can be inserted into the locking rings 1a so that the two lenses 1 can be joined together. A plurality of flanges 2a are disposed at inner sides of two ends of the joining ring 2. The flanges 2a can be locked with the lock rings 1a to adjust the distance between the two lenses 1. The two lock rings 1b at the left and right sides of the two lenses 1 are connected by a head band 4. Two ends of the head band 4 can be inserted into the lock rings 1b at the left and right sides of the lenses 1. Two distal ends of the head band 4 have two lock devices 4a, respectively. The lock devices 4a can prevent the head band 4 from detaching from the lock rings 1b at the left and right sides of the lenses 1. The lock devices 4a at the distal ends of the head band 4 can also be used to adjust the degree of tightness of the swimming glasses.

The outer side of the lens 1 forms a ring face 1c, which joins a pad 3 of soft material. A plurality of enhancement structures 3b are formed in the midst of a groove 3a in the pad 3. The enhancement structures 3b are alternately disposed in the groove 3a of the pad 3 to enhance the resilience of the swimming glasses and to elevate the position of the swimming glasses for preventing the nose bridge from contacting with the hard joining ring 2 of the swimming glasses. The outer edge of the groove 3a also has a ring face 3c, which is to be fixed on the skin of eye socket. The ring face 3c is also made of soft material to enhance comfort of wearing.

Please also refer to FIGS. 4 to 7. When the swimming glasses are worn, it is necessary to first sleeve the head band 4 of the swimming on the head and then adjust the two lenses 1 made by extrusion of hard and transparent material to the skins of eye sockets and let the ring faces 3c in the pads 3 of the swimming glasses contact tightly with the skins of eye sockets. Next, the head band 4 is pulled to a proper position of the back of the head. The swimming glasses can thus achieve airtight effect.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. An improved pad structure of swimming glasses, comprising:
two lenses with adjacent sides thereof extending to form lock rings, left and right sides of said two lenses also extending to form lock rings, an outer side of said lens forming a ring face;
a joining ring joined with said lock rings at adjacent sides of said two lenses, inner sides of two ends of said joining ring having a plurality flanges for adjusting the distance between said two lenses;
two pads each having a plurality of enhancement structures formed in the midst of a groove in said pad, an outer edge of said pad forming a ring face; and
a head band whose two ends can be inserted into said lock rings at the left and right sides of said two lenses, either distal end of said head band being inserted into a lock device.

2. The improved pad structure of swimming glasses as claimed in claim 1, wherein said pad is integrally formed of soft material.

3. The improved pad structure of swimming glasses as claimed in claim 1, wherein said enhancement structures are alternately disposed in said groove.
